(19) United States
(12) Patent Application Publication
(10) Pub. No.: US 2006/0222678 A1
(43) Pub. Date: Oct. 5, 2006

(54) Biomcompatible implant coated with biocompatible fluor-hydroxyapatite and a coating method of the same

(76) Inventors: Hyoun-Ee Kim, Seoul (KR); Hae-Won Kim, Gyeongbuk (KR); Sung-Min Chung, Seoul (KR); Sang-Wan Shin, Seoul (KR); Joon-Bong Park, Seoul (KR); Ee-Hyung Woo, Seoul (KR); Baek-Su Lee, Seoul (KR)

Correspondence Address: LADAS & PARRY, 26 WEST 61ST STREET, NEW YORK, NY, 10023, US

(21) Appl. No.: 10/556,639

(22) PCT Filed: Jul. 24, 2003
(86) PCT No.: PCT/KR03/01489

§ 371 Date: Nov. 10, 2005

(30) Foreign Application Priority Data
May 21, 2003 (KR) 10-2003-0032134
May 21, 2003 (KR) 10-2003-0032165

(51) Int. Cl. A61F 2/00; B05D 3/02

(52) U.S. Cl. 424/423; 427/2.24

(57) ABSTRACT

The present invention relates to a biocompatible implant coated with a biocompatible fluor-hydroxyapatite and a coating method of the same, and in particular to a method for coating a hydroxyapatite(HA) and a fluor-hydroxyapatite on a biocompatible implant Ti metal substrate having an excellent biocompatibility and mechanical property and a biocompatible implant coated based on the above method. It is possible to maximize of an apatite itself and a biocompatible activation characteristic of a substrate in such a manner that an apatite is coated on a titanium substrate having a high mechanical physical property by a sol gel method. It is possible to adjust a biocompatible activation based on a solubility difference of two kinds of apatites by coating a double layer of a hydroxyapatite and a fluor-hydroxyapatite.

### BACKGROUND OF THE INVENTION

1. Field of the invention

The present invention relates to a biocompatible implant coated with a biocompatible fluor-hydroxyapatite and a coating method of the same, and in particular to a method for coating a hydroxyapatite(HA) and a fluor-hydroxyapatite on a biocompatible implant titanium(Ti) metal substrate having an excellent biocompatibility and mechanical property and a biocompatible implant coated based on the above method.

2. Description of Related Art

A human body may corrode a certain metal therein. A biocompatibility implant titanium(Ti) is widely used as a biocompatible material for an implant in a dental field as well as an orthopedic surgery based on its excellent biocompatibility which is nearest an environment of a bone of a human body, an excellent mechanical property, and a no-harm to a human body in a reaction with a biocompatible tissue.

Recently, there are many attempts for physically and chemically improving a surface property of the same for enhancing an osseointegration. Among the above attempts, a process for coating an apatite is generally used. A hydroxyapatite which is used as a coating layer has a very similar crystal and chemical characteristic with respect to a hard tissue such as a bone, tooth, etc. of a human body. Therefore, when it is implanted into a living body, it does not make any trouble with a biocompatible tissue and a harmful reaction and has an excellent compatibility with a surrounding tissue.

The chemical formula of an apatite which is a ceramic coating layer for a biocompatible implant of the present invention may be expressed as follows in the formula (1).
Ca₁₀(PO₄)₆Z₂ (1)

Where Z represents OH, F, Cl or a compound of the same. The apatite consisted of OH and F mainly exists in the nature. OH represents hydroxyapatite, and F represents fluorapatite. There is a difference in a chemical stability.

Generally, it is known that a fluorapatite has a better chemical stability compared to a hydroxyapatite. In addition, the fluorapatite and hydroxyapatite are known to form a solid solution in the whole range based on an inter-substitution of operation groups OH and F for thereby forming a fluor-hydroxyapatite. As the amount of substitution of fluorine is increased, a chemical safety is enhanced.

In particular, in a dental medical field, there are many reports concerning the effect of fluorine ion itself. When it is used as a repairing structure material of teeth, an addition of a fluorine ion is directed to removing tartar and to enhancing a crystalline and plays an important role for forming a bone.

A certain effort is proceeded for substituting a damaged tooth or bone in a human body tissue using a biocompatibility and bioactivation of apatite. However, the apatite is known to have a disadvantage that a mechanical strength and fracture toughness are bad. Therefore, the apatite is not good as a hard tissue material of a human body which requires a high mechanical strength and fracture toughness such as an artificial dental implant or hip joint. The apatite is limitedly used for a portion which does not need a mechanical strength such as a bone in an inner ear.

In order to overcome the above described mechanical problems of the apatite, there are many attempts for coating an apatite on a metal or ceramic which has an excellent mechanical property as a coating layer. However, among the methods which are most widely used, a TPS(Titanium Plasma Spray) method is used. However, since this method is implemented at a very high temperature (6,000˜15,000° C.), the phase of the apatite is easily decomposed, so that it is impossible to obtain a pure and uniform composition. In addition, it is impossible to enhance an adhering strength with a titanium substrate by forming a thin coating layer of 50˜200 μm. Therefore, an exfoliation occurs in an interface with respect to a titanium substrate, and a roughness of surface is high. Therefore, there is a negative effect in a formation in grooves.

In addition, according to a result of the research, it is known that as the roughness of the surface is increased, an osseointegration is increased. Therefore, as a means for increasing a surface roughness, a sandblasting method is used. A blasting method in which a titania is used as a medium is most widely used.

Since a desired roughness is not achieved based on only a blasting method in which a titania is used, a method using Al₂O₃ is developed. However, the method using alumina has an advantage in increasing the roughness of surface, but it is not obviously known whether the alumina existing in the surface of an implant affects a human biocompatible or not. Therefore, the above method is not actually used for a clinical purpose.

In addition, an acid etching method is used. Since the method does not achieve a desired roughness of a surface based on only an acid etching process, it is preferred that the above method is used together with the blasting method. However, in this case, the process is complicated.

### SUMMARY OF THE INVENTION

Accordingly, it is an object of the present invention to provide a biocompatible implant coated with a fluor-hydroxyapatite for a biocompatible and a coating method of the same.

It is another object of the present invention to provide a method for coating a hydroxyapatite(HA) and a fluor-hydroxyapatite on a biocompatible implant titanium metal substrate having an excellent biocompatibility and a mechanical property.

In the present invention, an apatite sol is prepared using a sol-gel method for obtaining a good quality coating film according to the present invention.

To achieve the above objects, in a surface processing method of an implant for a biocompatible, there is provided a fluor-hydroxyapatite coating method for a biocompatible implant which includes a step for preparing a hydroxyapatite sol, a step for preparing a fluor-hydroxyapatite sol, a step for coating the hydroxyapatite sol and fluor-hydroxyapatite sol on a titanium implant, and a step for heat-treating a titanium substrate for a biocompatible implant coated with a titania.

### DETAILED DESCRIPTION OF PREFFERED EMBODIMENTS

The preferred embodiments of a titanium surface processing method for a biocompatible implant according to the present invention will be described with reference to the accompanying drawings.

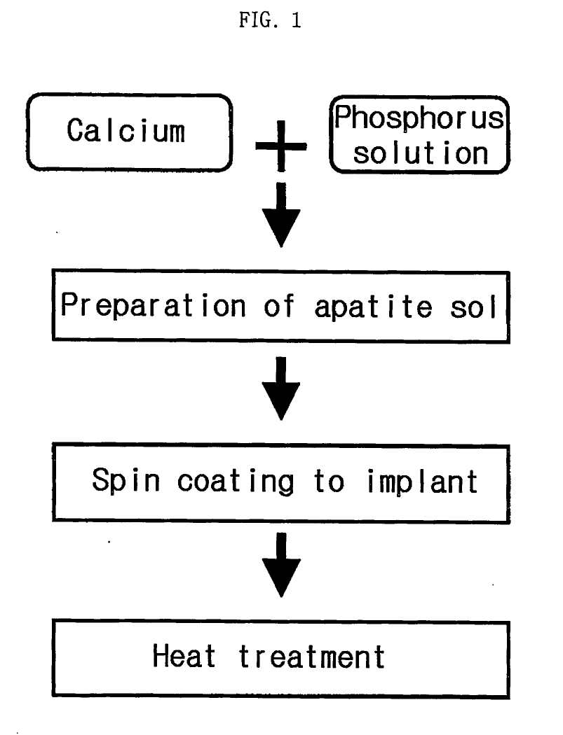

FIG. 1 is a view illustrating a flow chart of a coating process according to the present invention.

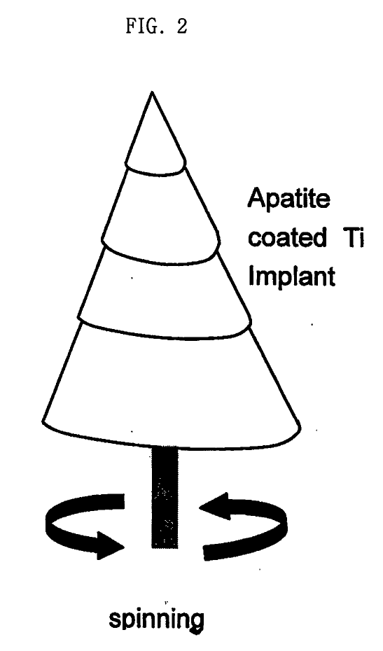

FIG. 2 is a view for describing a sol gel spin method for coating an apatite on a titanium for a biocompatible implant according to the present invention. As shown therein, an apatite sol is uniformly coated on a surface of a biocompatible implant based on a fast spin method.

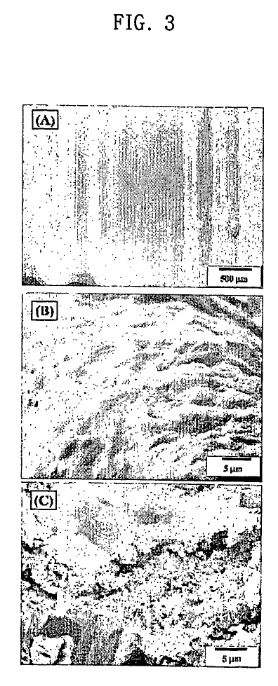

FIG. 3 is a picture taken using a scanning electron microscope(SEM) for illustrating a shape that an apatite is coated on a surface of a biocompatible implant titanium fabricated using a sol gel spin method according to the present invention. The process is performed in such a manner that a spin coating process is performed at 4,000 rpm for 60 seconds using a hydroxyapatite sol of 0.5 mol, and then a heating treatment process is performed. FIG. 3A is a low magnification surface picture with respect to a shape that a hydroxyapatite is coated. As shown therein, it is known that a hydroxyapatite coating layer is uniformly coated on a surface of a dental implant in a screw shape. FIG. 3B is a high magnification surface picture with respect to a shape coated with a hydroxyapatite. As shown therein, a hydroxyapatite coating layer is a dense and thin film and has a shape of an implant surface. FIG. 3C is a picture of a high magnification cur surface with respect to a shape coated with a hydroxyapatite. As shown therein, it is known that a thickness of a coating layer is about 1 μm.

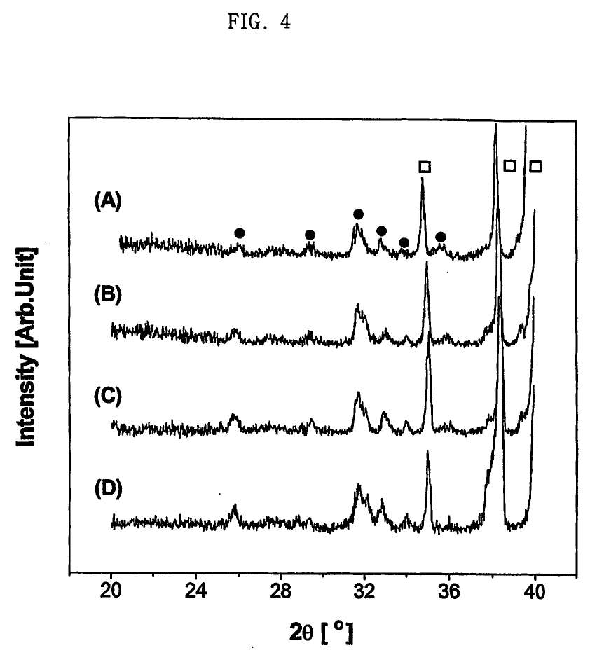

FIG. 4 is a graph obtained through a X-ray diffraction analysis with respect to the images formed after a hydroxyapatite and fluor-hydroxyapatite coating layer formed on a surface of a titanium fabricated according to the present invention are heat-treated for one hour at a temperature of 500° C. In this case, it is shown that a typical apatite crystalline was formed.

FIG. 5 is a graph illustrating dissolution degrees of a hydroxyapatite and fluor-hydroxyapatite coating layer formed on a surface of a titanium fabricated according to the present invention and shows a result of an analysis of the amount of a calcium ion eluted from a coating layer. The ion elution speed of the hydroxyapatite coating layer is fastest, and a 75 mol % fluor-hydroxyapatite coating layer is slowest, and a 25 mol % and 50 mol % fluor-hydroxyapatite coating layer is an intermediate speed therebetween. Namely, it is known that as the amount of substitution of fluorine ions is increased, an ion elution speed of the coating layer is decreased. The implant coated with an apatite material having a difference in the ion elution speed is adapted to a portion which needs a specific biocompatible activation in an actual biocompatible, so that it is impossible to implement an excellent biocompatibility.

FIG. 6 is a view illustrating a multiplication aspect of a cell after a MG63 cell is cultivated for 5 days for checking a cell reaction characteristic of a hydroxyapatite and a fluor-hydroxyapatite coating layer formed on a surface of a titanium fabricated according to the present invention. As shown therein, it is known that cells are well grown on all apatite coating layers.

FIG. 7 is a view illustrating a measurement of a differentiation degree of cells growing on an apatite coating layer. An activation degree of an alkaline phosphatase(ALP) is measured after the cells are grown for 10 days. The differentiation of the cells represents an activation of cells and a functional characteristic of a bone formation as a step after multiplication in a formation bone. The ALP activation is an important index which represents a cell differentiation. As a contrast group, a pure titanium is used. In the case that the coating is done using an apatite, a certain ALP activation degree which is largely higher than a pure titanium is obtained. The above result represents that it is possible to increase a biocompatibility by coating an apatite on a titanium. In particular, in the case of a fluor-hydroxyapatite, a cell differentiation degree similar with a coating layer of a hydroxyapatite is obtained. Therefore, the above has shown a possibility in use as an implant biocompatible material.

### EXAMPLES

The examples of the present invention will be described in detail.

### Example 1

The step for fabricating a hydroxyapatite sol includes a step in which Ca(NO₃)₂.4H₂O which is a material of a calcium are dissolved in an ethanol C₂H₅OH and are agitated for thereby preparing a calcium solution, a step in which P(CH₃CH₂O)₃ which is a material of a phosphorus and a distilled water(H₂O) are dissolved in an ethanol C₂H₅OH and are agitated for thereby preparing a phosphorus solution, a step in which a calcium solution and a phosphorus solution are mixed and agitated, and a step in which the above solution are aged.

The calcium solution and phosphorus solution are characterized in that calcium and phosphorus are mixed at a mol ratio of 1.67. The above mixture is aged at a room temperature for 60 hours through 80 hours and then is aged again at a temperature of 35° C. through 45° C. for 20 hours through 30 hours.

### Example 2

The step for preparing a fluor-hydroxyapatite sol includes a step in which Ca(NO₃)₂.4H₂O which is a material of a calcium are dissolved in an ethanol C₂H₅OH and are agitated for thereby preparing a calcium solution, a step in which P(CH₃CH₂O)₃ which is a material of a phosphorus and a distilled water(H₂O) are dissolved in an ethanol C₂H₅OH and are agitated for thereby preparing a phosphorus solution, a step in which NH₄F is added into the phosphorus solution, a step in which the calcium solution and phosphorus solution are mixed and agitated, and a step in which the above solution is aged.

The step in which NH₄F is added is characterized in that the ratio with respect to F⁻ and OH⁻ is 25 mol %, 50 mol % and 75 mol %.

### Example 3

The step for coating an apatite sol on a titanium substrate includes a step in which an apatite sol is applied to a biocompatible implant titanium substrate for thereby wetting the titanium substrate, a step in which a spin coating process is performed using a spin coating unit, a step in which the titanium substrate coated with an apatite sol is dried, and a step in which the titanium substrate is heat-treated.

The spin coating process is performed in such a manner that a spinning operation is performed for 10 seconds through 30 seconds at 2,500 through 3,500 rpm. The above heat treatment is performed for 1 through 2 hours at 400° C. through 600° C.

As described above, in the present invention, it is possible to enhance a biocompatibility by coating an apatite on a biocompatible implant titanium for thereby implementing a good osseointegration.

In addition, since the apatite coating uses a sol gel method, it is possible to simplify a process and to implement a thin and uniform film.

The sol gel apatite coating method is capable of implementing a uniform thin film coating on a surface of a dental root shaped implant of a complicated shape through a spin coating. Therefore, the present invention is well adapted to a coating process of other biocompatible implants having a complicated shape.

In addition, the fluor-hydroxyapatite and hydroxyapatite coating later according to the present invention has a certain difference in the solution speed, so that it is possible to effectively control a biocompatible activation of an implant. It is possible to implement a functional gradient coating in which a biocompatible activation has a uniform difference by fabricating a hydroxyapatite as an outer later and a fluor-hydroxyapatite layer as an inner layer.

It is possible to implement a beneficial biocompatible characteristic that a fluorine ion has in such a manner that a fluorine ion is contained in a biocompatible implant surface.

It is expected that the demand of a biocompatible implant fabricated according to the present invention will be increased, and an enough supply may be implemented based on its mass production characteristic.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

1. In a surface processing method of an implant for a biocompatible, a fluor-hydroxyapatite coating method for a biocompatible implant, comprising the steps of:
a step for preparing a hydroxyapatite sol;
a step for preparing a fluor-hydroxyapatite sol;
a step for coating the hydroxyapatite sol and fluor-hydroxyapatite sol on a titanium implant; and
a step for heat-treating a titanium substrate for a biocompatible implant coated with a titania.

2. The method of claim 1, wherein said step for preparing a hydroxyapatite sol includes the steps of:
a step in which Ca(NO₃)₂.4H₂O which is a material of a calcium are dissolved in an ethanolC₂H₅OH and are agitated for thereby preparing a calcium solution;
a step in which P(CH₃CH₂O)₃ which is a material of a phosphorus and a distilled water(H₂O) are dissolved in an ethanol C₂H₅OH and are agitated for thereby preparing a phosphorus solution;
a step in which a calcium solution and a phosphorus solution are mixed and agitated; and
a step in which the above solution are aged.

3. The method of claim 2, wherein a mixture solution of the calcium solution and phosphorus solution are characterized in that calcium and phosphorus are mixed at a mol ratio of 1.67.

4. The method of claim 2, wherein said mixture solution is aged at a room temperature for 60 hours through 80 hours and then is aged again at a temperature of 35° C. through 45° C. for 20 hours through 30 hours.

5. The method of claim 1, wherein said step for preparing a fluor-hydroxyapatite sol includes the steps of:
a step in which Ca(NO₃)₂.4H₂O which is a material of a calcium are dissolved in an ethanol C₂H₅OH and are agitated for thereby preparing a calcium solution;
a step in which P(CH₃CH₂O)₃ which is a material of a phosphorus and a distilled water (H₂O) are dissolved in an ethanol C₂H₅OH and are agitated for thereby preparing a phosphorus solution;
a step in which NH₄F is added into the phosphorus solution;
a step in which the calcium solution and phosphorus solution are mixed and agitated; and a step in which the above solution is aged.

6. The method of claim 5, wherein said step in which NH₄F is added is characterized in that the ratio with respect to F⁻ and OH⁻ is 25 mol %, 50 mol % and 75 mol %.

7. The method of claim 1, wherein said for coating an apatite sol on a titanium substrate includes a step in which an apatite sol is applied to a biocompatible implant titanium substrate for thereby wetting the titanium substrate, a step in which a spin coating process is performed using a spin coating unit, a step in which the titanium substrate coated with an apatite sol is dried, and a step in which the titanium substrate is heat-treated.

8. The method of claim 7, wherein a spin coating process is performed in such a manner that a spinning operation is performed for 10 seconds through 30 seconds at 2,500 through 3,500 rpm.

9. The method of claim 7, wherein said drying process is performed for 5 through 7 hours at a temperature of 70° C. through 90° C.

10. The method of claim 7, wherein said heat treatment is performed for 1 through 2 hours at 400° C. through 600° C.

11. A biocompatible implant coated with a fluor-hydroxyapatite which is fabricated by the method of claim 1.

12. A biocompatible implant coated with a fluor-hydroxyapatite which is fabricated by the method of claim 2.

13. A biocompatible implant coated with a fluor-hydroxyapatite which is fabricated by the method of claim 3.

14. A biocompatible implant coated with a fluor-hydroxyapatite which is fabricated by the method of claim 4.

15. A biocompatible implant coated with a fluor-hydroxyapatite which is fabricated by the method of claim 5.

16. A biocompatible implant coated with a fluor-hydroxyapatite which is fabricated by the method of claim 6.

17. A biocompatible implant coated with a fluor-hydroxyapatite which is fabricated by the method of claim 7.

18. A biocompatible implant coated with a fluor-hydroxyapatite which is fabricated by the method of claim 8.

19. A biocompatible implant coated with a fluor-hydroxyapatite which is fabricated by the method of claim 9.

20. A biocompatible implant coated with a fluor-hydroxyapatite which is fabricated by the method of claim 10.
